(19) United States
(12) Patent Application Publication
(10) Pub. No.: US 2020/0056156 A1
(43) Pub. Date: Feb. 20, 2020

(54) CELL POPULATION COMPRISING MESENCHYMAL STEM CELLS, METHOD FOR PRODUCING THE SAME, AND PHARMACEUTICAL COMPOSITION

(71) Applicant: KANEKA CORPORATION, Osaka-shi, Osaka (JP)

(72) Inventors: Keita INO, Hyogo (JP); Yuta KITA, Hyogo (JP)

(73) Assignee: KANEKA CORPORATION, Osaka-shi, Osaka (JP)

(21) Appl. No.: 16/500,261

(22) PCT Filed: Apr. 3, 2018
(86) PCT No.: PCT/JP2018/014324

§ 371 Date: Oct. 2, 2019

(30) Foreign Application Priority Data
Apr. 3, 2017 (JP) 2017-073982

(51) Int. Cl. C12N 5/0775; A61K 35/28

(52) U.S. Cl. CPC C12N 5/0668; C12N 2509/00; A61K 35/28

(57) ABSTRACT

An object of the present invention is to provide a cell population comprising mesenchymal stem cells having high angiogenic capacity, a method for producing the same, a mesenchymal stem cell having high angiogenic capacity, and a pharmaceutical composition comprising the cell population. Also provided are methods for producing a cell population comprising mesenchymal stem cells, in particular methods comprising obtaining a cell population having the following cell characteristics (a) and (b): (a) the proportion of CDH6-positive mesenchymal stem cells is 30% or more in the cell population; and (b) the relative expression level of ADAM19 gene to the expression level of SDHA gene is 3.0 or more in the cell population.

### TECHNICAL FIELD

The present invention relates to a method for producing a cell population comprising mesenchymal stem cells. Furthermore, the present invention relates to a cell population comprising mesenchymal stem cells and a pharmaceutical composition comprising the cell population.

### BACKGROUND ART

Mesenchymal stem cells, also called as mesenchymal stromal cells, are somatic stem cells that have been reported to be present in the bone marrow, adipose tissue, tooth pulp, and the like. Recently, it has been revealed that these cells also exist in the fetal appendage including the placenta, umbilical cord, and fetal membrane. Mesenchymal stem cells are capable of differentiating into bones, cartilage, and fats, etc. and thus have been gaining attention as a promising cell source in regenerative medicine.

Mesenchymal stem cells have the ability to secrete cytokines that promote angiogenesis as well as differentiation capacity. Thus, it has been reported that treatment of cardiac failure, myocardial infarction and the like may be attained by transplanting cell sheets containing mesenchymal stem cells to the epicardial surface of the heart, or administering a cell suspension containing mesenchymal stem cells to the coronary artery.

Patent Literature 1 describes a method for producing amniotic mesenchymal cell composition, a method for cryopreserving the amniotic mesenchymal cell composition, and a therapeutic agent. Particularly, this document discloses that the cryopreserved amniotic mesenchymal cells can be produced as a cell preparation optimized for transplantation, by cryopreserving a mixture comprising amniotic mesenchymal cells in a solution containing 5 to 10% by mass of dimethyl sulfoxide and containing 5 to 10% by mass of hydroxyethyl starch or 1 to 5% by mass of dextran.

Patent Literature 2 describes a method for preparing an amniotic mesenchymal stem cell population, comprising the steps of: (D) collecting a cell population of mesenchymal cells from the amnion of a mammal; (E) inoculating the collected cell population at a cell concentration of 400 to 35000 cells/cm², followed by initial culture for 2 to 3 days; (F) inoculating the cultured cells at 1/5000 or more and less than 1/10 of the cell concentration of the initial culture, and repeating subculture three or four times with medium replacement twice a week; and (G) maintaining the culture of the cells in the same culture dish until confluent when a colony of cells having a fusiform shape is formed in the subculture.

Patent Literature 3 describes a method for treating cardiac failure in a patient, comprising transplanting a transplantation cell sheet containing mesenchymal stem cells into the heart of the patient.

### CITATION LIST

### Patent Literature

### Patent Literature 1: JP Patent Publication (Kokai) No. 2015-61520 A (2015)

Patent Literature 2: International Publication No. WO2013/077428
Patent Literature 3: International Publication No. WO2006/080434.

### SUMMARY OF INVENTION

### Technical Problem

In recent years, mesenchymal stem cells derived from a fetal appendage have been found to be a heterogeneous cell population comprising various cells having different differentiation capacities, proliferative capacities, and cytokine producing capacities. For producing a stable quality of cell preparation, it is necessary to prepare a purified highly homogeneous cell population.

Patent Literature 1 discloses that cryopreserved amniotic mesenchymal cells can be produced as a cell preparation optimized for transplantation, by cryopreserving a mixture comprising amniotic mesenchymal cells in a particular cryopreservation solution to prevent decrease in the survival rate of amniotic mesenchymal cells after thawing. However, this literature makes no mention about the selective preparation of mesenchymal stem cells having a particular excellent characteristic from among mesenchymal stem cells, specifically, the selective preparation of a cell population rich in mesenchymal stem cells having high angiogenic capacity by utilizing the characteristics of mesenchymal stem cells as an index. In Patent Literature 2, a mesenchymal stem cell population having high proliferative capacity and differentiation capacity is prepared by inoculating cells at a low density. However, this literature neither describes nor suggests selection of a cell population rich in mesenchymal cells having high angiogenic capacity by utilizing the characteristics of mesenchymal stem cells comprised in a mesenchymal stem cell population as an index. Patent Literature 3 also discloses that a sheet of mesenchymal stem cells for transplantation was prepared by culturing mesenchymal stem cells isolated from the adipose tissue by collagenase treatment on a temperature-responsive culture dish until confluent and then changing temperature to 32° C. or less. However, there is no description or suggestion about the use of the characteristics of mesenchymal stem cells contained in the mesenchymal stem cell population as an indication to selectively acquire a cell population rich in mesenchymal cells having high angiogenic capacity.

An object of the present invention is to provide cell population comprising mesenchymal stem cells having high angiogenic capacity and a method for producing the same.

### Solution to Problem

As a result of intensive studies in order to achieve the above object, the present inventors found that a cell population comprising cells collected from the fetal appendage contains mesenchymal stem cells that are CDH6 positive and highly express ADAM 19 gene; and cell population comprising mesenchymal stem cells having the above cell characteristics highly express genes (ANGPT1 gene, VEGFC gene, and HBEGF gene) encoding angiogenesis-related cytokines and having high angiogenic capacity. The present invention has been completed on the basis of these findings.

Specifically, the present specification provides the following aspects of the invention:

[1] A method for producing a cell population comprising mesenchymal stem cells, the method comprising obtaining a cell population having the following cell characteristics (a) and (b):

(a) the proportion of CDH6-positive mesenchymal stem cells is 30% or more in the cell population; and

(b) the relative expression level of ADAM19 gene to the expression level of SDHA gene is 3.0 or more in the cell population.

[2] A cell population comprising mesenchymal stem cells, the cell population having the following cell characteristics (a) and (b):
(a) the proportion of CDH6-positive mesenchymal stem cells is 30% or more in the cell population; and
(b) the relative expression level of ADAM19 gene to the expression level of SDHA gene is 3.0 or more in the cell population.
[3] The cell population according to [2], wherein the mesenchymal stem cells are derived from a fetal appendage.
[4] A pharmaceutical composition comprising the cell population according to [2] or [3] and a pharmaceutically acceptable vehicle.
[5] The pharmaceutical composition according to [4], wherein a single dose of the mesenchymal stem cells to a human is 10⁹ cells/kg body weight or less.
[6] The pharmaceutical composition according to [4] or [5], wherein the pharmaceutical composition is an injectable preparation.
[7] The pharmaceutical composition according to [4] or [5], wherein the pharmaceutical composition is a preparation for transplanting a cell aggregate or sheet-like structure.
[8] The pharmaceutical composition according to any one of [4] to [7], wherein the pharmaceutical composition is a therapeutic agent for a disease selected from ischemic diseases, lower-limb ischemia, renal ischemia, pulmonary ischemia, ischemic heart disease, coronary heart disease, myocardial infarction, angina pectoris, cardiac failure, cardiomyopathy, valvular disease, cerebrovascular ischemia, stroke, cerebral infarction, intracerebral hematoma, and cerebrovascular paralysis.
[9] The cell population according to [2] or [3], wherein at least one of the following is satisfied: the relative expression level of ANGPT1 gene to the expression level of SDHA gene is 0.2 or more; the relative expression level of VEGFC gene to the expression level of SDHA gene is 1.0 or more; and the relative expression level of HBEGF gene to the expression level of SDHA gene is 2.0 or more.
[10] A cell population obtained by the production method according to [1].
[11] A use of the cell population according to [2] or [3] for the manufacture of a pharmaceutical composition.
[12] The use according to [11], wherein the pharmaceutical composition is a pharmaceutical composition where a single dose of the mesenchymal stem cells to a human is 10⁹ cells/kg body weight or less.
[13] The use according to [11] or [12], wherein the pharmaceutical composition is an injectable preparation.
[14] The use according to [11] or [12], wherein the pharmaceutical composition is a preparation for transplanting a cell aggregate or sheet-like structure.
[15] The use according to any one of [11] to [14], wherein the pharmaceutical composition is a therapeutic agent for a disease selected from ischemic diseases, lower-limb ischemia, renal ischemia, pulmonary ischemia, ischemic heart disease, coronary heart disease, myocardial infarction, angina pectoris, cardiac failure, cardiomyopathy, valvular disease, cerebrovascular ischemia, stroke, cerebral infarction, intracerebral hematoma, and cerebrovascular paralysis.
[16] The cell population according to [2] or [3] for use in the treatment of a disease.
[17] The cell population according to [16], wherein a single dose of the mesenchymal stem cells to a human is 10⁹ cells/kg body weight or less.
[18] The cell population according to [16] or [17], wherein the cell population is an injectable preparation.
[19] The cell population according to [16] or [17], wherein the cell population is a preparation for transplanting a cell aggregate or sheet-like structure.
[20] The cell population according to any one of [16] to [19], wherein the disease is selected from ischemic diseases, lower-limb ischemia, renal ischemia, pulmonary ischemia, ischemic heart disease, coronary heart disease, myocardial infarction, angina pectoris, cardiac failure, cardiomyopathy, valvular disease, cerebrovascular ischemia, stroke, cerebral infarction, intracerebral hematoma, and cerebrovascular paralysis.
[21] A method for treating a disease, comprising administering the cell population according to [2] or [3] to a patient in need of treatment.
[22] The method for treating a disease according to [21], wherein a single dose of the mesenchymal stem cells to a human is 10⁹ cells/kg body weight or less.
[23] The method for treating a disease according to [21] or [22], wherein the method is an injectable preparation.
[24] The method for treating a disease according to [21] or [22], wherein the method is a preparation for transplanting a cell aggregate or sheet-like structure.
[25] The method for treating a disease according to any one of [21] to [24], wherein the disease is selected from ischemic diseases, lower-limb ischemia, renal ischemia, pulmonary ischemia, ischemic heart disease, coronary heart disease, myocardial infarction, angina pectoris, cardiac failure, cardiomyopathy, valvular disease, cerebrovascular ischemia, stroke, cerebral infarction, intracerebral hematoma, and cerebrovascular paralysis.
[26] A composition comprising the cell population according to [2] or [3] and a vehicle.

### Advantageous Effects of Invention

According to the present invention, a cell population comprising mesenchymal stem cells with high expression levels of genes encoding angiogenesis-related cytokines (ANGPT1 gene, VEGFC gene, and HBEGF gene) can be obtained using the positive rate of surface antigens CDH6 and the expression level of ADAM19 gene as indices. Thus, cell population comprising mesenchymal stem cells highly expresses genes encoding angiogenesis-related cytokines (ANGPT1 gene, VEGFC gene, and HBEGF gene) and thus is considered to have high angiogenic capacity, and for example, can be used as a pharmaceutical composition for the treatment of ischemic diseases, cardiac failure, stroke, and the like.

### DESCRIPTION OF EMBODIMENTS

Embodiments of the present invention will be specifically described below. The descriptions are intended to facilitate understanding of the principles of the present invention, and therefore, the scope of the present invention is not limited to the embodiments. Other embodiments, to which a person skilled in the art may make modification(s), are also included in the scope of the invention.

### [1] Explanation of Terms

The term “fetal appendage” used herein refers to a fetal membrane, a placenta, an umbilical cord, and amniotic fluid. In addition, the term “fetal membrane” refers to a fetal sac containing fetal amniotic fluid, which comprises an amnion, a chorion, and a decidua in that order from the inside. Among them, the amnion and the chorion are originated from the fetus. The term “amnion” refers to a transparent thin membrane with few blood vessels, which is located in the most inner layer of the fetal membrane. The inner layer (also called as epithelial cell layer) of the amnion is covered with a layer of epithelial cells having a secretory function and secretes amniotic fluid. The outer layer (also called as extracellular matrix layer, which corresponds to the stroma) of the amnion comprises mesenchymal stem cells.

The term “mesenchymal stem cells (MSCs)” used herein refers to stem cells that satisfy the definition described below, and are used interchangeably with “mesenchymal stromal cells”. The term “mesenchymal stem cells” is also referred to as “MSCs”.

Definition of Mesenchymal Stem Cells

i) Adherence to plastic in standard medium under culture conditions.

ii) Positive for surface antigens CD105, CD73, and CD90, and negative for surface antigens CD45, CD34, CD11b, CD79alpha, CD19, and HLA-DR.

The term “mesenchymal stem cell population” used herein means a cell population comprising mesenchymal stem cells. Examples of the form thereof include, but not particularly limited to, cell pellets, cell aggregates, cell-floated liquids and cell suspensions.

The term “amniotic mesenchymal stem cells” used herein refers to mesenchymal stem cells derived from the amnion, and are used interchangeably with “amniotic mesenchymal stromal cells”. The term “amniotic mesenchymal stem cells” used herein is also referred to as “amniotic MSCs”.

The term “angiogenic capacity” used herein refers to the ability to form new blood vessels. Angiogenic capacity can be evaluated by the expression level(s) of ANGPT1 gene, VEGFC gene and/or HBEGF gene, and the evaluation method can be carried out by the microarray analysis described in the examples described later.

The phrase “proportion of CDH6-positive-mesenchymal stem cells” used herein refers to the proportion of cells positive for the surface antigen analyzed by flow cytometry as described in Examples mentioned later. The phrase “proportion of CDH6-positive-mesenchymal stem cells” used herein is also referred to as “positive rate”.

### [2] Cell Population Comprising Mesenchymal Stem Cells

The cell population comprising mesenchymal stem cells, provided by the present invention, is characterized in that:

(a) the proportion of CDH6-positive mesenchymal stem cells is 30% or more in the cell population; and
(b) the relative expression level of ADAM19 gene to the expression level of SDHA gene is 3.0 or more in the cell population.

The abbreviation CDH6 stands for Cadherin 6, which is a member of the cadherin superfamily. The abbreviation ADAM19 stands for ADAM metallolopetidase domain 19.

The cell population comprising mesenchymal stem cells, provided by the present invention, forms a cell population comprising mesenchymal cells having high angiogenic capacity when the following conditions are satisfied: the proportion of CDH6-positive-mesenchymal stem cells is 30% or more and the relative expression level of ADAM19 gene to the expression level of SDHA gene is 3.0 or more. Hence, in the present invention, said conditions can be utilized as indices for the formation of a cell population having high angiogenic capacity.

The proportion of CDH6-positive-mesenchymal stem cells in the cell population may be 31% or more, 32% or more, 33% or more, 34% or more, 35% or more, 36% or more, 37% or more, 38% or more, 39% or more, or 40% or more.

The surface antigen marker (CDH6) can be detected by any detection method known in the art. Examples of the method for detecting the surface antigen marker include, but not limited to, flow cytometry and cell staining. When cells that emit stronger fluorescence as compared with a negative control (isotype control) are detected in flow cytometry using a fluorescently labeled antibody, the cells are determined to be “positive” for the marker. Any antibody known in the art can be used as the fluorescently labeled antibody. Examples thereof include, but not limited to, antibodies labeled with fluorescein isothiocyanate (FITC), phycoerythrin (PE), allophycocyanin (APC), or the like. When cells that are stained in cell staining or emit fluorescence are observed under a microscope, the cells are determined to be “positive” for the marker. The cell staining may be cell immunostaining using an antibody, or may be non-immune cell staining using no antibody.

Specifically, the proportion of cells positive (positive rate) for surface antigen marker (CDH6) can be determined using an extended flow cytometry dot-plot analysis by the following procedures (1) to (8):

(1) When the cells of interest are adherent cells, the adherent cells are detached from a plastic culture vessel using trypsin-EDTA (Thermo Fisher Scientific Inc.) and then collected by centrifugation. When the cells of interest are nonadherent cells, the cells are collected by centrifugation.
(2) The cells are fixed with 4% paraformaldehyde and then washed with phosphate buffer (PBS), followed by being prepared as a cell suspension in 2% BSA/PBS at 1.0×10⁶ cells/mL. The cell suspension is dispensed in 100 μL aliquots.
(3) The dispensed cell suspensions are centrifuged. Then, 100 μL of 0.5% BSA/PBS is added to each of the obtained cell pellets, followed by addition of antibodies against the respective surface antigen markers or the isotype control antibodies thereof. When CDH6, a surface antigen marker, is analyzed, mouse IgG (H+L) is subsequently added as a secondary antibody. Each reaction solution is vortexed and then allowed to stand at 4° C. for 20 minutes.
(4) After adding 0.5% BSA/PBS and washing the cells by centrifugation, the cells are suspended in 0.5% BSA/PBS and filtered through a cell strainer (35-μm nylon mesh filter) (Corning Inc./Product number: 352235).
(5) The cell suspension obtained by filter filtration is analyzed by All Event 10000 on a BD Accuri™ C6 Flow Cytometer (Becton, Dickinson and Company).
(6) The measurement results are plotted as dots on the horizontal axis as SSC (side scattered light) (numerical range: 0 or more and 16777215 or less) and the vertical axis as fluorescence intensity of the dye labeled to the antibody (numerical range: 10¹ or more and 10^{7.2} or less).
(7) In the dot plot diagram, all regions (gates) are selected such that the cell population with higher fluorescence intensity is 1.0% or less among all cells measured with the isotype control antibodies.
(8) The percentage of cells contained in the gate selected in (7) among all cells measured with the antibody against the surface antigen marker is calculated.

Examples of the timing to detect the surface antigen marker include, but not particularly limited to, immediately after separation of cells from a biological sample, during a culture step, after purification in the culture step, immediately after the Nth passage (N represents an integer of 1 or more), during maintenance culture, before cryopreservation, after thawing, and before formulation as a pharmaceutical composition.

The cell population comprising mesenchymal stem cells, provided by the present invention, satisfies:

a relative expression level of ADAM19 gene to the expression level of SDHA gene is 3.0 or more.

The relative expression level of AMDAM 19 gene to the expression level of SDHA gene may be 3.5 or more, 4.0 or more, or 4.5 or more.

As a method of measuring the relative expression level of ADAM19 gene to the expression level of SDHA gene, a measurement using a microarray can be used. Specifically, the microarray can be performed by the procedures (1) to (5) described below. Here, the following procedures (3) to (5) can be entrusted to RIKEN GENESIS CO., LTD.

(1) When the target cells are adherent cells, the adherent cells are detached non-enzymatically using a cell scraper (manufactured by Corning Inc.) from the plastic culture vessel, and the cells are then collected by centrifugation. When the target cells are nonadherent cells, the cells are collected by centrifugation.
(2) After stable storage of cells using a RNA stabilization reagent (RNAlater, manufactured by Thermo Fisher Scientific Inc.), total RNA is extracted and purified using a RNA extraction kit (RNeasy Plus Mini kit, manufactured by QIAGEN ltd.).
(3) cDNA is synthesized by reverse transcription using the purified total RNA as a template. Then, the synthesized cDNA is further transcribed to cRNA by in vitro transcription and labeled with biotin.
(4) Biotin-labeled cRNA is added to a hybridization buffer and subjected to hybridization for 16 hours on Human GeneGenome U133A 2.0 Array (manufactured by Affymetrix, Inc.). The hybridized product was washed with GeneChip Fluidics Station 450 (manufactured by Affymetrix, Inc.), and stained with phycoerythrin, followed by being scanned with GeneChip Scanner 3000 7G (manufactured by Affymetrix, Inc.), and subjected to image analysis with AGCC (Affymetrix GeneChip Command Console Software, manufactured by Affymetrix, Inc.) and then digitized using Affymetrix Expression Console (manufactured by Affymetrix, Inc.).
(5) Numerical data files are compared and analyzed using analysis software GeneSpring GX (manufactured by Agilent Technologies, Inc.). The relative expression level of ADAM19 gene to the expression level of SDHA gene in each cell is calculated.

The sequence of SDHA (Succinate dehydrogenase complex, subunit A) gene is registered as ID: 6389 in the gene database of National Center for Biotechnology Information.

ADAM19 (ADAM metallopeptidase domain 19) gene is registered as ID: 8728 in the gene database of National Center for Biotechnology Information.

Angiogenic capacity can be evaluated by the expression level(s) of one or more genes selected from ANGPT1 gene, VEGFC gene, or HBEGF gene. Angiogenic capacity may be evaluated as the relative expression level of each of the above genes to the expression level of SDHA gene by the microarray analysis described in the examples below.

The sequence of ANGPT1 (Angiopoietin 1) gene is registered as ID: 284 in the gene database of National Center for Biotechnology Information.

The sequence of VEGFC (Vascular endothelial growth factor C) gene is registered as ID: 7424 in the gene database of National Center for Biotechnology Information.

The sequence of HBEGF (Heparin binding-epidermal growth factor-like growth factor) gene is registered as ID: 1839 in the gene database of National Center for Biotechnology Information.

The relative expression level of ANGPT1 gene to the expression level of SDHA gene may be preferably 0.2 or more, 0.3 or more, or 0.4 or more.

The relative expression level of VEGFC gene to the expression level of SDHA gene may be preferably 1.0 or more, 1.1 or more, 1.2 or more, 1.3 or more, 1.4 or more, 1.5 or more, 1.6 or more, 1.7 or more, 1.8 or more, 1.9 or more, 2.0 or more, 2.1 or more, or 2.2 or more.

The relative expression level of HBEGF gene to the expression level of SDHA gene may be preferably 2.0 or more, 2.1 or more, 2.2 or more, 2.3 or more, 2.4 or more, 2.5 or more, or 2.6 or more.

The timing to measure the gene expression level mentioned above is but not particularly limited to, immediately after separation of cells from a biological sample, during a culture step, after purification in the culture step, immediately after the Nth passage (N represents an integer of 1 or more), during maintenance culture, before cryopreservation, after thawing, and before formulation as a pharmaceutical composition.

The origin of mesenchymal stem cells is not particularly limited, but for example, mesenchymal stem cells derived from fetal appendage, bone marrow, fat, or tooth pulp can be used. Mesenchymal stem cells are preferably mesenchymal stem cells derived from a fetal appendage, more preferably mesenchymal stem cells derived from the amnion.

The mesenchymal stem cell population of the present invention may be preserved in a frozen state until immediately before use. The mesenchymal stem cell population may comprise any component, in addition to the mesenchymal stem cells. Examples of such a component can include, but not limited to, salts, polysaccharides (e.g., hydroxyethyl starch (HES) and dextran), proteins (e.g., albumin), DMSO, amino acids, and medium components (e.g., components contained in RPMI1640 medium).

The cell population of the present invention may be provided as a composition in combination with a vehicle. As the vehicle, preferably a liquid vehicle (e.g., a medium or a pharmaceutical acceptable vehicle as described later) can be used.

The cell population of the present invention may include any number of amniotic mesenchymal cells. The cell population of the present invention may include, but not limited to, not less than or no more than 1×10¹, 2×10¹, 5×10¹, 1×10², 2×10², 5×10², 1×10³, 2×10³, 5×10³, 1×10⁴, 2×10⁴, 5×10⁴, 1×10⁵, 2×10⁵, 5×10⁵, 1×10⁶, 2×10⁶, 5×10⁶, 1×10, 2×10⁷, 5×10⁷, 1×10⁸, 2×10⁸, 5×10⁸, 1×10⁹, 2×10⁹, 5×10⁹, 1×10¹⁰, 2×10¹⁰, 5×10¹⁰, 1×10¹¹, 2×10¹¹, 5×10¹¹, 1×10¹², 2×10¹², or 5×10¹² of mesenchymal stem cells.

### [3] Method for Producing a Cell Population Comprising Mesenchymal Stem Cells

The method for producing a cell population comprising mesenchymal stem cells according to the present invention is a method comprising obtaining a cell population having the following cell characteristics (a) and (b):

(a) the proportion of CDH6-positive mesenchymal stem cells is 30% or more in the cell population; and

(b) the relative expression level of ADAM19 gene to the expression level of SDHA gene is 3.0 or more in the cell population.

In other words, the method for producing a cell population comprising mesenchymal stem cells according to the present invention is a method comprising the step of preparing a cell population comprising mesenchymal stem cells under the conditions for retaining the above cell characteristics (a) and (b). The above conditions of (a) and (b) serve as indices for the formation of a cell population comprising mesenchymal stem cells having high angiogenic capacity. The production method of the present invention is not particularly limited as long as the indices are satisfied.

The production method of the present invention may comprise a cell population obtainment step of obtaining a cell population comprising mesenchymal stem cells by the enzyme treatment of a sample (e.g., the fetal appendage such as the amnion) comprising mesenchymal stem cells.

The amnion consists of an epithelial cell layer and an extracellular matrix layer. The latter layer comprises amniotic MSCs. The amniotic epithelial cells are characterized in that they express epithelial cadherin (E-cadherin: CD324) and an epithelial cell adhesion factor (EpCAM: CD326) like other epithelial cells, while the amniotic MSCs do not express such epithelial-specific surface antigen markers. Thus, these cells can be easily distinguished by flow cytometry. The cell population obtainment step may comprise the step of obtaining the amnion by cesarean section.

The cell population comprising mesenchymal stem cells according to the present invention is preferably a cell population obtained by treating a sample comprising an epithelial cell layer and an extracellular matrix layer collected from the fetal appendage with at least collagenase.

The enzyme treatment of the sample collected from the fetal appendage (preferably a sample comprising an epithelial cell layer and a extracellular matrix layer) is preferably a treatment with an enzyme (or a combination of enzymes) that can release mesenchymal stem cells contained in the extracellular matrix layer of the fetal appendage, and does not degrade the epithelial cell layer. Examples of such an enzyme can include, but not particularly limited to, collagenase and/or metalloproteinase. Examples of the metalloproteinase can include, but not particularly limited to, thermolysin and/or dispase, which is metalloproteinase that cleaves nonpolar amino acids at their N-terminal sides.

The active concentration of the collagenase is preferably 50 PU/ml or higher, more preferably 100 PU/ml or higher, further preferably 200 PU/ml or higher, further preferably 300 PU/ml or higher, and further preferably 400 PU/ml or higher. The active concentration of the collagenase is, but is not particularly limited to, for example, 1000 PU/ml or lower, 900 PU/ml or lower, 800 PU/ml or lower, 700 PU/ml or lower, 600 PU/ml or lower, or 500 PU/ml or lower. In this context, PU (protease unit) is defined as the amount of the enzyme that degrades 1 μg of FITC-collagen in 1 minute at 30° C. at pH 7.5.

The active concentration of the metalloproteinase (e.g., thermolysin and/or dispase) is preferably 50 PU/ml or higher, more preferably 100 PU/ml or higher, further preferably 200 PU/ml or higher, further preferably 300 PU/ml or higher, and further preferably 400 PU/ml or higher. Also, the active concentration of the metalloproteinase is preferably 1000 PU/ml or lower, more preferably 900 PU/ml or lower, further preferably 800 PU/ml or lower, further preferably 700 PU/ml or lower, further preferably 600 PU/ml or lower, and further preferably 500 PU/ml or lower. In this context, PU (protease unit) in an aspect of using dispase as the metalloproteinase is defined as the amount of the enzyme that releases an amino acid corresponding to 1 μg tyrosine from casein lactate in 1 minute at 30° C. at pH 7.5. In the concentration range of the enzyme described above, mesenchymal stem cells contained in the extracellular matrix layer can be efficiently released while prevented from being contaminated with epithelial cells contained in the epithelial cell layer of the fetal appendage. The preferred combination of the concentrations of the collagenase and/or the metalloproteinase can be determined by the microscopic observation of the fetal appendage after the enzyme treatment, or the flow cytometry of the obtained cells.

It is preferred to treat the fetal appendage with collagenase and metalloproteinase in combination at the same time, from the viewpoint of efficiently recovering live cells. In this case, thermolysin and/or dispase can be used as the metalloproteinase, though the metalloproteinase is not limited thereto. Mesenchymal stem cells can be conveniently obtained by treating the fetal appendage only once with an enzyme solution containing collagenase and metalloproteinase. The treatment at the same time in one operation can reduce the risk of contamination by bacteria, viruses, and the like.

For the enzyme treatment of the fetal appendage, it is preferred to immerse, in the enzyme solution, the amnion washed using a washing solution such as physiological saline or Hank's balanced salt solution, and perform the treatment with stirring using stirring means. A stirrer or a shaker can be used as such stirring means from the viewpoint of efficiently releasing mesenchymal stem cells contained in the extracellular matrix layer of the fetal appendage, though the stirring means is not limited thereto. The stirring rate is not particularly limited and is, for example, 5 rpm or more, 10 rpm or more, 20 rpm or more, 30 rpm or more, 40 rpm or more or 50 rpm or more when using a stirrer or a shaker. Also, the stirring rate is not particularly limited and is, for example, 100 rpm or less, 90 rpm or less, 80 rpm or less, 70 rpm or less or 60 rpm or less when using a stirrer or a shaker. The enzyme treatment duration is not particularly limited and is, for example, 10 minutes or longer, 20 minutes or longer, 30 minutes or longer, 40 minutes or longer, 50 minutes or longer, 60 minutes or longer, 70 minutes or longer, 80 minutes or longer or 90 minutes or longer. Also, the enzyme treatment duration is not particularly limited and is, for example, 6 hours or shorter, 5 hours or shorter, 4 hours or shorter, 3 hours or shorter, 2 hours or shorter, 110 minutes or shorter, 100 minutes or shorter. The enzyme treatment temperature is not particularly limited and is, for example, 15° C. or higher, 16° C. or higher, 17° C. or higher, 18° C. or higher, 19° C. or higher, 20° C. or higher, 21° C. or higher, 22° C. or higher, 23° C. or higher, 24° C. or higher, 25° C. or higher, 26° C. or higher, 27° C. or higher, 28° C. or higher, 29° C. or higher, 30° C. or higher, 31° C. or higher, 32° C. or higher, 33° C. or higher, 34° C. or higher, 35° C. or higher or 36° C. or higher. Also, the enzyme treatment temperature is not particularly limited and is, for example, 40° C. or lower, 39° C. or lower, 38° C. or lower or 37° C. or lower.

In the production method of the present invention, if desired, the released mesenchymal stem cells can be separated and/or collected from the enzyme solution containing the released mesenchymal stem cells by a known method such as a filter, centrifugation, a hollow fiber separation membrane, or a cell sorter. Preferably, the enzyme solution containing the released mesenchymal stem cells is filtered through a filter. In an aspect of filtering the enzyme solution through a filter, only the released cells pass through the filter, whereas an undegraded epithelial cell layer remains on the filter without passing through the filter. Therefore, not only can the released mesenchymal stem cells be easily separated and/or collected, but the risk of contamination by bacteria, viruses, and the like can be reduced. Examples of the filter can include, but not particularly limited to, mesh filters. The pore size (mesh size) of the mesh filter is not particularly limited and is, for example, 40 μm or larger, 50 μm or larger, 60 μm or larger, 70 μm or larger, 80 μm or larger, or 90 μm or larger. Also, the pore size of the mesh filter is not particularly limited and is, for example, 200 μm or smaller, 190 μm or smaller, 180 μm or smaller, 170 μm or smaller, 160 μm or smaller, 150 μm or smaller, 140 μm or smaller, 130 μm or smaller, 120 μm or smaller, 110 μm or smaller, or 100 μm or smaller. The filtration rate is not particularly limited. When the pore size of the mesh filter falls within the range described above, the enzyme solution containing the mesenchymal stem cells can be filtered by free fall. This can prevent decrease in cell survival rate.

Nylon is preferably used as a material for the mesh filter. A tube containing a 40 μm, 70 μm, 95 μm or 100 μm nylon mesh filter such as a Falcon cell strainer, which is widely used for research purposes, is available. Alternatively, medical mesh cloth (nylon and polyester) used for hemodialysis and the like is available. Further, an arterial filter used for extracorporeal circulation (polyester mesh filter, pore size: 40 μm or larger and 120 μm or smaller) is also available. A mesh made of any other material, for example, a stainless-steel mesh filter, may be used.

Preferably, the mesenchymal stem cells are allowed to pass through a filter in a free fall motion. It is also possible to force the cells to pass through a filter by suction using a pump or the like. In this case, minimum necessary pressurization is desirable in order to avoid damage of the cells.

The mesenchymal stem cells that have passed through the filter can be collected by centrifugation after dilution of the filtrate with a medium or balanced salt buffer solution by two times or more. Examples of the balanced salt buffer solution that can be used include, but not limited to, Dulbecco's phosphate-buffered saline (DPBS), Earle's balanced salt solution (EBSS), Hank's balanced salt solution (HBSS), and phosphate-buffered saline (PBS).

The cell population obtained in the cell population obtainment step is prepared under the following conditions:

(a) the proportion of CDH6-positive mesenchymal stem cells is 30% or more in the cell population; and

(b) the relative expression level of ADAM19 gene to the expression level of SDHA gene is 3.0 or more in the cell population. The conditions are useful as indices for obtaining a cell population comprising mesenchymal stem cells having high angiogenic capacity. The preparation method is not particularly limited as long as the indices are satisfied. Examples of such a method can include for example, obtaining a cell population satisfying the above (a) with a cell sorter, and then selecting a cell population satisfying the above (b) from the obtained cell population; and selecting a cell population satisfying the above (b), and then obtaining a cell population satisfying the above (a) with a cell sorter from the obtained cell population. In addition, examples of preparation methods that satisfy the index include culturing a cell population under the conditions satisfying the above (a) and (b).

Examples of the culture method that satisfies the indices can include the step of repeating a plurality of times the inoculation of the cell population onto an uncoated plastic culture vessel at a density of 400 to 5,000 cells/cm², followed by culture. The density of the cell population for inoculation is more preferably 500 cells/cm² or more, further preferably 600 cells/cm² or more, further preferably 700 cells/cm² or more, further preferably 800 cells/cm² or more, further preferably 900 cells/cm² or more, further preferably 1000 cells/cm² or more, further preferably 1100 cells/cm² or more, further preferably 1200 cells/cm² or more, further preferably 1300 cells/cm² or more, further preferably 1400 cells/cm² or more, further preferably 1500 cells/cm² or more, further preferably 1600 cells/cm² or more, further preferably 1700 cells/cm² or more, further preferably 1800 cells/cm² or more, further preferably 1900 cells/cm² or more, and further preferably 2000 cells/cm² or more. The density of the cell population for inoculation is more preferably 4800 cells/cm² or less, further preferably 4600 cells/cm² or less, further preferably 4400 cells/cm² or less, further preferably 4200 cells/cm² or less, further preferably 4000 cells/cm² or less, further preferably 3800 cells/cm² or less, further preferably 3600 cells/cm² or less, further preferably 3400 cells/cm² or less, further preferably 3200 cells/cm² or less, further preferably 3000 cells/cm² or less, further preferably 2800 cells/cm² or less, further preferably 2600 cells/cm² or less, further preferably 2400 cells/cm² or less, and further preferably 2200 cells/cm² or less.

Examples of the other culture methods that satisfy the indices can include the step of repeating a plurality of times the inoculation of the cell population onto a plastic culture vessel coated with a coating agent at a density of 400 to 5,000 cells/cm², followed by culture. Preferred density conditions for the inoculation of the cell population are similar to those described above.

Examples of the coating agent can include, but not limited to, extracellular matrix, fibronectin, vitronectin, osteopontin, laminin, entactin, collagen I, collagen II, collagen III, collagen IV, collagen V, collagen VI, gelatin, poly-L-ornithine, poly-D-lysine, and Matrigel(registered trademark) matrix.

Examples of the other culture methods that satisfy the indices include culturing with addition of basic fibroblast growth factor (bFGF) to the basal medium for use in the culture. The concentration of the basic fibroblast growth factor is preferably 2 ng/mL or more, further preferably 4 ng/mL or more, further preferably 6 ng/mL or more, further preferably 8 ng/mL or more, or further preferably 10 ng/mL or more. The concentration of the basic fibroblast growth factor is preferably 20 ng/mL or less, further preferably 18 ng/mL, or less 16 ng/mL or less, further preferably 14 ng/mL or less, or further preferably 12 ng/mL or less. The timing of adding the basic fibroblast growth factor is not particularly limited and is, for example, at the beginning of the culture step, during a culture step, after purification in the culture step, immediately after the Nth passage (N represents an integer of 1 or more), during maintenance culture, before cryopreservation, or after thawing.

Examples of the culture period of one culture process can include 4 to 10 days, and, more specifically, can include 4 days, 5 days, 6 days, 7 days, 8 days, 9 days and 10 days.

The medium for use in the culture can be prepared by utilizing any liquid medium for animal cell culture as a basal medium and, if necessary, appropriately adding other components (serum, a serum replacement reagent, a growth factor, etc.) thereto.

Examples of the basal medium that can be used include, but not particularly limited to, media such as BME medium, BGJb medium, CMRL1066 medium, Glasgow MEM medium, improved MEM zinc option medium, IMDM medium (Iscove's modified Dulbecco's medium), Medium 199 medium, Eagle MEM medium, αMEM (alpha modification of minimum essential medium eagle) medium, DMEM medium (Dulbecco's modified Eagle's medium), Ham's F10 medium, Hams' F12 medium, RPMI 1640 medium, Fischer's medium, and mixed medium thereof (e.g., DMEM/F12 medium (Dulbecco's modified Eagle's medium/nutrient mixture F-12 Ham)).

Alternatively, the medium for use in the culture may be a commercially available serum-free medium. Examples thereof include, but not particularly limited to, STK1 and STK2 (DS Pharma Biomedical Co., Ltd.), EXPREP MSC Medium (BioMimetics Sympathies Inc.), and Corning stemgro human mesenchymal stem cell medium (Corning Inc.).

Examples of other components to be added to the basal medium include albumin, serum, serum replacement reagents and growth factors. In an aspect of adding albumin to the basal medium, the concentration of albumin is preferably higher than 0.05% and 5% or lower. Also, in an aspect of adding serum to the basal medium, the concentration of serum is preferably 5% or higher. In an aspect of adding a growth factor, a reagent (heparin, etc.) for stabilizing the growth factor in the medium may be added in addition to the growth factor or the growth factor may be stabilized with a gel, a polysaccharide, or the like in advance, and then the stabilized growth factor may be added to the basal medium.

The culture of mesenchymal stem cells can be performed by, for example, the following process: first, a cell suspension is centrifuged, the supernatant is removed, and the obtained cell pellet is suspended in a medium. Next, the cells are inoculated to an uncoated plastic culture vessel and cultured using a medium in an environment of a CO₂ concentration of 3% or higher and 5% or lower at 37° C. until 95% or less confluence. Examples of the medium can include, but not limited to, αMEM, M199, and media based thereon. The cells obtained by the culture as described above are cells cultured once.

The cells cultured once can be further passaged and cultured, for example, as follows: first, the cells cultured once are dissociated from the plastic culture vessel by treatment with ethylenediaminetetraacetic acid (EDTA) followed by treatment with trypsin. Next, the obtained cell suspension is centrifuged, the supernatant is removed, and the obtained cell pellet is suspended in a medium. Finally, the cells are inoculated to an uncoated plastic culture vessel and cultured using a medium in an environment of a CO₂ concentration of 3% or higher and 5% or lower at 37° C. until 95% or less confluence. Examples of the medium can include, but not limited to, αMEM, M199, and media based thereon. The cells obtained by the passage and the culture as described above are cells passaged once. Cells passaged N times can be obtained by similar passage and culture (N represents an integer of 1 or more). The lower limit of passage number N is, for example, 1 or more, preferably 2 or more, more preferably 3 or more, further preferably 4 or more, further preferably 5 or more, further preferably 6 or more, further preferably 7 or more, further preferably 8 or more, further preferably 9 or more, further preferably 10 or more, further preferably 11 or more, further preferably 12 or more, further preferably 13 or more, further preferably 14 or more, further preferably 15 or more, further preferably 16 or more, further preferably 17 or more, further preferably 18 or more, further preferably 19 or more, further preferably 20 or more, and further preferably 25 or more, from the viewpoint of producing the cells at a large scale. Also, the upper limit of passage number N is, for example, preferably 50 or less, 45 or less, 40 or less, 35 or less, or 30 or less, from the viewpoint of suppressing cell senescence.

According to the production method of the present invention, a cell population comprising mesenchymal stem cells having high angiogenic capacity can be obtained. The lower limit of the obtained cell count per batch of culture (cell count obtained per unit surface area and per unit number of culture days) differs depending on an inoculated cell count, an inoculation density, etc. and is, for example, 1.0×10⁵ (cells/cm²/day) or more, 2.0×10⁵ (cells/cm²/day) or more, 3.0×10⁵ (cells/cm²/day) or more, 4.0×10⁵ (cells/cm²/day) or more, 5.0×10⁵ (cells/cm²/day) or more, 6.0×10⁵ (cells/cm²/day) or more, 7.0×10⁵ (cells/cm²/day) or more, 8.0×10⁵ (cells/cm²/day) or more, 9.0×10⁵ (cells/cm²/day) or more or 10.0×10⁵ (cells/cm²/day) or more. Also, the upper limit of the obtained cell count per batch of culture is not particularly limited and is, for example, 10.0×10⁸ (cells/cm²/day) or less, 9.0×10⁸ (cells/cm²/day) or less, 8.0×10⁸ (cells/cm²/day) or less, 7.0×10⁸ (cells/cm²/day) or less, 6.0×10⁸ (cells/cm²/day) or less, 5.0×10⁸ (cells/cm²/day) or less, 4.0×10⁸ (cells/cm²/day) or less, 3.0×10⁸ (cells/cm²/day) or less, 2.0×10⁸ (cells/cm²/day) or less or 1.0×10⁸ (cells/cm²/day) or less.

According to the production method of the present invention, cell population comprising mesenchymal stem cells having high angiogenic capacity can be obtained. The mesenchymal stem cells obtained by the production method of the present invention are thereby culturable preferably up to 40 days or later, and more preferably up to 45 days or later, up to 50 days or later, up to 55 days or later, up to 60 days or later, up to 65 days or later, up to 70 days or later, up to 75 days or later, up to 80 days or later, up to 85 days or later, up to 90 days or later, up to 95 days or later, up to 100 days or later, up to 105 days or later, or up to 110 days or later, after the start of in vitro culture.

The mesenchymal stem cells obtained by the production method of the present invention are also culturable up to a population doubling level of preferably 10 or more, and more preferably 15 or more, 20 or more, 25 or more, 30 or more, 35 or more, 40 or more, 45 or more, or 50 or more, after the start of in vitro culture.

The production method of the present invention may comprise an identification step of identifying a cell population comprising mesenchymal stem cells having high angiogenic capacity by utilizing, as indices, the following conditions:

(a) the proportion of CDH6-positive mesenchymal stem cells is 30% or more in the cell population; and
(b) the relative expression level of ADAM19 gene to the expression level of SDHA gene is 3.0 or more in the cell population.

Means for identifying the cell population comprising mesenchymal stem cells are preferably flow cytometry and a microarray.

The proportion of CDH6-positive-mesenchymal stem cells (positive rate) in the cell population can be measured according to the procedure described in paragraph 0028 using an extended flow cytometry dot-plot analysis.

The relative expression level of ADAM19 gene to the expression level of SDHA gene can be measured according to the procedure described in paragraph 0032 using a microarray.

The timing to perform the above identification is, but not particularly limited to, immediately after separation of cells from a biological sample, during a culture step, after purification in the culture step, immediately after the Nth passage (N represents an integer of 1 or more), during maintenance culture, before cryopreservation, after thawing, and before formulation as a pharmaceutical composition.

The production method of the present invention can comprise the step of selective separation of the identified cell population after identifying the cell population comprising mesenchymal stem cells by utilizing the above (a) and (b) as indices. Means for selectively separating the identified cell population is not particularly limited and is, for example, fractionation of the cell populations by a cell sorter and purification of the cell population by culture.

The production method of the present invention may also comprise the step of cryopreserving the cell population comprising mesenchymal stem cells. In an aspect comprising the step of cryopreserving the cell population, the cell population may be thawed, and then, if necessary, identified, separated, collected and/or cultured. Alternatively, the cell population may be thawed and then directly used.

Examples of the means for cryopreserving cell population comprising mesenchymal stem cells include, but not particularly limited to, program freezers, deep freezers, and immersing in liquid nitrogen. When a program freezer is used, the temperature for freezing is preferably −30° C. or lower, −40° C. or lower, −50° C. or lower, −60° C. or lower, −70° C. or lower, −80° C. or lower, −90° C. or lower, −100° C. or lower, −110° C. or lower, −120° C. or lower, −130° C. or lower, −140° C. or lower, −150° C. or lower, −160° C. or lower, −170° C. or lower, −180° C. or lower, −190° C. or lower, or −196° C. (liquid nitrogen temperature) or lower. When a program freezer is used, the freezing rate for freezing is, for example, preferably −1° C./min, −2° C./min, −3° C./min, −4° C./min, −5° C./min, −6° C./min, −7° C./min, −8° C./min, −9° C./min, −10° C./min, −11° C./min, −12° C./min, −13° C./min, −14° C./min or −15° C./min. When using a program freezer as such freezing means, the temperature can be lowered to a temperature between −50° C. or higher and −30° C. or lower (e.g., −40° C.) at a freezing rate of, for example, −2° C./min or more and −1° C./min or less, and further lowered to a temperature of −100° C. or higher and −80° C. or lower (e.g., −90° C.) at a freezing rate of −11° C./min or more and −9° C./min or less (e.g., −10° C./min). When using immersing in liquid nitrogen as such freezing means, the temperature can be rapidly lowered to a temperature of −196° C. to freeze, and then cryopreservation can be performed in liquid nitrogen (gas phase).

For freezing by the freezing means, the cell population may be frozen in a state contained in any preservation container. Examples of such a preservation container include, but not limited to, cryotubes, cryovials, bags for freezing, and infusion bags.

For freezing by the freezing means, the cell population may be frozen in any cryopreservation solution. Examples of such a cryopreservation solution include, but not limited to, commercially available cryopreservation solutions, such as CryoNovo (Akron Biotechnology, LLC.), MSC Freezing Solution (Biological Industries, Inc.) and CryoStor (HemaCare Inc.).

The cryopreservation solution can contain polysaccharides at a predetermined concentration. The preferred concentration of polysaccharides is, for example, 1% by mass or higher, 2% by mass or higher, 3% by mass or higher, 4% by mass or higher, 5% by mass or higher, 6% by mass or higher, 7% by mass or higher, 8% by mass or higher, 9% by mass or higher, 10% by mass or higher, 11% by mass or higher, or 12% by mass or higher. Alternatively, the preferred concentration of polysaccharides is, for example, 40% by mass or lower, 35% by mass or lower, 30% by mass or lower, 25% by mass or lower, 20% by mass or lower, 19% by mass or lower, 18% by mass or lower, 17% by mass or lower, 16% by mass or lower, 15% by mass or lower, 14% by mass or lower, or 13% by mass or lower. Examples of polysaccharides can include, but not limited to, hydroxylethyl starch (HES) and dextran (e.g., Dextran 40).

The cryopreservation solution can contain dimethylsulfoxide (DMSO) at a predetermined concentration. The preferable concentration of DMSO is, for example, 1% by mass or higher, 2% by mass or higher, 3% by mass or higher, 4% by mass or higher, 5% by mass or higher, 6% by mass or higher, 7% by mass or higher, 8% by mass or higher, or 9% by mass or higher. Alternatively, the preferable concentration of DMSO is, for example, 20% by mass or lower, 19% by mass or lower, 18% by mass or lower, 17% by mass or lower, 16% by mass or lower, 15% by mass or lower, 14% by mass or lower, 13% by mass or lower, 12% by mass or lower, 11% by mass or lower, or 10% by mass or lower.

The cryopreservation solution may contain albumin at a predetermined concentration larger than 0% by mass. The preferable concentration of the albumin is, for example, preferably 0.5% by mass or higher, 1% by mass or higher, 2% by mass or higher, 3% by mass or higher, 4% by mass or higher, 5% by mass or higher, 6% by mass or higher, 7% by mass or higher or 8% by mass or higher. Also, the concentration of the albumin is, for example, preferably 40% by mass or lower, 35% by mass or lower, 30% by mass or lower, 20% by mass or lower, 15% by mass or lower, 10% by mass or lower or 9% by mass or lower. Examples of the albumin can include, but not limited to, bovine serum albumin, mouse albumin, and human albumin.

### [4] Pharmaceutical Composition

The cell population comprising mesenchymal stem cells according to the present invention can be used as a pharmaceutical composition. Specifically, the present invention provides a pharmaceutical composition comprising the cell population according to the present invention and a pharmaceutically acceptable vehicle.

The pharmaceutical composition of the present invention is preferably a liquid preparation, more preferably an injectable liquid preparation.

The pharmaceutical composition of the present invention can be used as a cell therapy agent, for example, a therapeutic agent for intractable disease(s).

The pharmaceutical composition of the present invention can be used as a therapeutic agent for a disease selected from a group consisting of ischemic diseases, lower-limb ischemia, renal ischemia, pulmonary ischemia, ischemic heart disease, coronary heart disease, myocardial infarction, angina pectoris, cardiac failure, cardiomyopathy, valvular disease, cerebrovascular ischemia, stroke, cerebral infarction, intracerebral hematoma, and cerebrovascular paralysis. The above disease can be treated by administering the pharmaceutical composition of the present invention to a treatment site in an amount such that the effect can be measured.

The present invention provides the cell population comprising mesenchymal stem cells according to the present invention for use in a pharmaceutical composition.

The present invention provides the cell population comprising mesenchymal stem cells according to the present invention for use in a cell therapy agent.

The present invention provides the cell population comprising mesenchymal stem cells according to the present invention for use in the treatment of a disease selected from a group consisting of ischemic diseases, lower-limb ischemia, renal ischemia, pulmonary ischemia, ischemic heart disease, coronary heart disease, myocardial infarction, angina pectoris, cardiac failure, cardiomyopathy, valvular disease, cerebrovascular ischemia, stroke, cerebral infarction, intracerebral hematoma, and cerebrovascular paralysis.

The present invention provides a method for transplanting cells to a patient or a subject, and a method for treating a disease in a patient or a subject, comprising the step of administering a therapeutically effective amount of the cell population comprising mesenchymal stem cells according to the present invention to the patient or the subject.

The present invention provides use of the cell population comprising mesenchymal stem cells according to the present invention for the manufacture of a pharmaceutical composition.

The present invention provides use of the cell population comprising mesenchymal stem cells according to the present invention for the manufacture of a cell therapy agent.

The present invention provides use of the cell population comprising mesenchymal stem cells according to the present invention for the manufacture of a therapeutic agent for a disease selected from a group consisting of ischemic diseases, lower-limb ischemia, renal ischemia, pulmonary ischemia, ischemic heart disease, coronary heart disease, myocardial infarction, angina pectoris, cardiac failure, cardiomyopathy, valvular disease, cerebrovascular ischemia, stroke, cerebral infarction, intracerebral hematoma, and cerebrovascular paralysis.

The dose of the pharmaceutical composition of the present invention is the amount of cells that allows a patient or a subject to whom the pharmaceutical composition has been administered to obtain therapeutic effects, compared with a patient or a subject to whom the pharmaceutical composition has not been administered. A specific dose can be appropriately determined depending on the form of administration, an administration method, intended use, and patient's or subject's age, body weight, and symptoms, and the like. A single dose of the mesenchymal stem cells to a human is not particularly limited and is, for example, 10⁴ cells/kg body weight or more, 10⁵ cells/kg body weight or more or 10⁶ cells/kg body weight or more. Also, a single dose of the mesenchymal stem cells to a human is not particularly limited and is, for example, 10⁹ cells/kg body weight or less, 10⁸ cells/kg body weight or less or 10⁷ cells/kg body weight or less.

Examples of the method for administering the pharmaceutical composition of the present invention include, but not particularly limited to, subcutaneous injection, intra-lymph nodal injection, intravenous injection, intraperitoneal injection, intrathoracic injection, direct localized injection, and direct localized transplantation. Examples of known methods for administering the pharmaceutical composition include intravenous injection, intravenous drip injection, local direct injection, local direct transplantation, and the like as described in Japanese Patent Publication (Kokai) No. 2015-61520 and Onken J E, et al., American College of Gastroenterology Conference 2006 Las Vegas, Nev., Abstract 121., Garcia-Olmo D, et al., Dis Colon Rectum 2005; 48: 1416-23. The pharmaceutical composition according to the present invention can also be administered by various methods described in these documents.

The pharmaceutical composition of the present invention may be used as an injectable preparation, a preparation for transplanting a cell aggregate or sheet-like structure, or a gel preparation mixed with any gel, for the purpose of treating other diseases.

The patient or the subject of the present invention is typically a human and may be other animals. Examples of other animals include, but not limited to, mammal such as dogs, cats, cows, horses, pigs, goats, sheep, monkeys (cynomolgus monkeys, rhesus monkeys, common marmosets, and Japanese monkeys), ferrets, rabbits, rodents (mice, rats, gerbils, guinea pigs, and hamsters), and birds such as chickens and quails.

The pharmaceutical composition of the present invention can be preserved in a frozen state until immediately before use. It can be used by rapidly thawing at 37° C. for administrating the pharmaceutical composition of the present invention to the patient or the subject.

The pharmaceutical composition of the present invention may comprise any component for use in the treatment of humans. Examples of such a component can include, but not limited to, salts, polysaccharides (e.g., HES and dextran), proteins (e.g., albumin), DMSO, amino acids, and medium components (e.g., components contained in RPMI1640 medium).

The pharmaceutical composition of the present invention may be a cell population comprising mesenchymal stem cells diluted with an infusion preparation used as a pharmaceutically acceptable vehicle. The term “infusion preparation (pharmaceutically acceptable vehicle)” used herein is not particularly limited as long as it is a solution used in the treatment of humans. Examples thereof include physiological saline, 5% glucose solution, Ringer's solution, lactated Ringer's solution, acetated Ringer's solution, starter solution (Solution I), rehydration solution (Solution II), maintenance infusion (Solution III), and postoperative recovery solution (Solution IV).

The pharmaceutical composition of the present invention may contain various additives for increasing preservation stability, sterility, isotonicity, absorbability and/or viscosity, such as emulsifiers, dispersants, buffers, preservatives, wetting agents, antimicrobial agents, antioxidants, chelating agents, thickeners, gelling agents, pH adjusters, and the like. Examples of the thickener include, but not limited to, HES, dextran, methylcellulose, xanthan gum, carboxymethylcellulose, hydroxypropyl cellulose, and the like. The concentration of the thickener can be optionally set according to the selected thickener, within the range of concentration that is safe when administered to the patient or the subject and achieves the desired viscosity.

The pH of the pharmaceutical composition of the present invention can be adjusted to almost neutral pH, for example, pH 6.5 or more or pH 7.0 or more, and/or pH 8.5 or lower or pH8.0 or lower, but not limited thereto.

The present invention will be specifically described with reference to the Examples below; however, the present invention is not limited to the Examples.

### EXAMPLES

### Comparative Example 1

### (Step 1: Collection of Amnion)

A fetal membrane and a placenta, which are the fetal appendages, were aseptically collected from a pregnant woman(donor) who was an elective cesarean section case after obtaining informed consent. The obtained fetal membrane and placenta were contained in a sterile tray containing physiological saline. An amnion was manually separated from the stump of the fetal membrane. The amnion was washed with a Hank's balanced salt solution (free of Ca and Mg) to remove attached blood and clots.

### (Step 2: Enzyme Treatment of Amnion and Collection of Amniotic MSCs)

The amnion was enzyme-treated by immersing the amnion comprising an epithelial cell layer and an extracellular matrix layer in a Hank's balanced salt solution (containing Ca and Mg) containing 300 PU/mL collagenase and 200 PU/mL dispase I, and shaking and stirring under conditions of 37° C., 90 minutes, and 50 rpm. The solution thus enzyme-treated was filtered through a nylon mesh having openings of 95 μm to remove undigested products of the amnion so as to collect a cell suspension containing amniotic MSCs. The obtained cell suspension was analyzed for the proportion of cells positive for the expression level of CD90, which is a surface antigen known as a typical positive marker of MSCs, using a flow cytometer. As a result, it was confirmed that the amniotic MSCs were able to be separated with high purity from the amnion.

The surface antigen analysis employed BD Accuri™ C6 Flow Cytometer from Becton, Dickinson and Company, and the measurement conditions involved analyzed cell count: 10,000 cells and flow rate setting: Slow (14 μL/min). FITC Mouse Anti-Human CD90 (Becton, Dickinson and Company/model number: 561969) was used as the antibody against the CD90 antigen, and, as an isotype control antibody, FITC Mouse IgG1, κ Isotype Control (Becton, Dickinson and Company/model number: 349041) was used.

### (Step 3: Cryopreservation of Amniotic MSCs)

The cell population obtained in the above section “Step 2: Enzyme treatment of amnion and recovery of amniotic MSCs” was suspended in BAMBANKER (LYMPHOTEC Inc.) so as to be 1.0×10⁷ cells/mL and then aliquoted into cryotubes. The cryotubes were placed in BICELL (freezing container) (NIHON FREEZER CO., LTD.) and stored at −80° C. for 12 hours, followed by being cryopreserved at liquid nitrogen temperature.

### (Step 4: Culture of Amniotic MSCs)

The cell population obtained in the above section “Step 3: Cryopreservation of amniotic MSCs” was inoculated to an uncoated plastic culture vessel and adherent cultured until subconfluent in αMEM (alpha modification of minimum essential medium Eagle) containing 10% fetal bovine serum (FBS) (inactivated) and 1× Antibiotic-Antimycotic (manufactured by Thermo Fisher Scientific Inc.). Then, the cells were dissociated using TrypLE Select (1×) (manufactured by Thermo Fisher Scientific Inc.). A ¼ amount of the cells was inoculated to an uncoated plastic culture vessel at the same scale as that of the preceding culture and thereby subcultured. Medium replacement was carried out with a frequency of twice a week. Thus, the subculture was continued.

### Example 1

### (Step 1: Collection of Amnion)

The amnion was obtained in the same manner as in Step 1 of Comparative Example 1 except that the fetal membrane and the placenta, which are the fetal appendages, were aseptically collected from a donor different from the donor of Step 1 of Comparative Example 1.

### (Step 2: Enzyme Treatment of Amnion and Collection of Amniotic MSCs)

A cell suspension containing the amniotic MSCs was collected by the same procedures as in step 2 of Comparative Example 1. The obtained cell suspension was analyzed for the proportion of cells positive for the expression of CD90, a surface antigen known as a typical positive marker of MSCs, using a flow cytometer in the same way as in Comparative Example 1. As a result, it was confirmed that amniotic MSCs were able to be separated with high purity from the amnion.

### (Step 3: Cryopreservation of Amniotic MSCs)

The cell population obtained in the above section “Step 2: Enzyme treatment of amnion and collection of amniotic MSCs” was cryopreserved by the same procedures as in Step 3 of Comparative Example 1.

### (Step 4: Culture of Amniotic MSCs)

The cell population obtained in the above section “Step 3: Cryopreservation of amniotic MSCs” was adherent cultured until subconfluent by the same procedures as in Step 4 of Comparative Example 1. Subculture was carried by the same procedures as in Step 4 of Comparative Example 1.

### <Analysis of CDH6 Expression>

For the cell populations of the 6th passage cultured in Comparative Example 1 and Example 1, the proportion of cells positive for CDH6 was measured using a flow cytometer.

In this assay, Anti-CDH6-Mouse Mono IgG1 (R&D Systems, Inc./model number: MAB2715) was used as a primary antibody against CDH6 antigen, Mouse Mono IgG1 (R&D Systems, Inc./model number: MAB002) was used as an isotype control antibody, and Mouse F(ab)₂ IgG (H+L) APC-conjugated Antibody (R&D Systems, Inc./model number: F0101B) was used as a secondary antibody against the primary antibody against CDH6 antigen and the isotype control antibody thereof.

The analysis results are shown in Table 1 below.

TABLE 1
Surface antigen analysis of each cell population
by flow cytometry (dot-plot analysis) (positive rate, %)

| | Cell population of | Cell population of |
|---|---|---|
| | Example 1 | Comparative Example 1 |
| CDH6 | 40.0 | 20.3 |

The positive rate of CDH6 was improved in the cell population of Example 1, compared to the cell population of Comparative Example 1. Furthermore, the proportion of CDH6-positive-mesenchymal stem cells is less than 30% (specifically, 20.3%) in the cell population of Comparative Example 1, while the proportion of CDH6-positive-mesenchymal stem cells were at least 30% (specifically, 40.0%) in the cell population of Example 1.

### <Analysis of ADMA19 Gene Expression>

For the cell populations of the 6th passage cultured in Comparative Example 1 and Example 1, the expressions of ADAM19 and SDHA genes were analyzed by the microarray analysis.

The microarray was performed by the following procedures (1) to (5). Here, the following procedures (3) to (5) were entrusted to and performed by RIKEN GENESIS CO., LTD.

(1) The cell populations of the 6th passage cultured in Comparative Example 1 and Example 1 were dissociated from the plastic culture vessel using a cell scraper (manufactured by Corning Inc.) and collected by centrifugation.
(2) RNAlater (manufactured by Thermo Fisher Scientific Inc.) was added to the obtained cell pellet to stably store RNA, followed by extraction and purification of total RNA using RNeasy Plus Mini kit (manufactured by QIAGEN ltd.).
(3) cDNA was synthesized by reverse transcription from 100 ng of total RNA. Then, cDNA was transcribed into cRNA by in vitro transcription and labeled with biotin (using 3′IVT PLUS Reagent Kit).
(4) Labeled cRNA 10.0 μg was added to a hybridization buffer and subjected to hybridization for 16 hours on Human GeneGenome U133A 2.0 Array (manufactured by Affymetrix, Inc.). The hybridized product was washed with GeneChip Fluidics Station 450 (manufactured by Affymetrix, Inc.) and stained with phycoerythrin, followed by being scanned with Gene Chip Scanner 3000 7G (manufactured by Affymetrix, Inc.) and subjected to image analysis with AGCC (Affymetrix GeneChip Command Console Software) (manufactured by Affymetrix, Inc.). The image data was quantified using Affymetrix Expression Console (Manufactured by Affymetrix, Inc.).
(5) The numerical data file was analyzed using analysis software GeneSpring GX (manufactured by Agilent Technologies, Inc.).

The expression level of ADAM19 gene was determined as a relative expression level to the expression level SDHA gene. The results are shown in Table 2 below.

TABLE 2
Analysis results of cell characteristics

| | | ADAM19 |
|---|---|---|
| Cell population of | Fluorescence intensity | 4830.6 |
| Example 1 | Relative expression level* | 4.9 |
| Cell population of | Fluorescence intensity | 2407.4 |
| Comparative Example 1 | Relative expression level* | 2.3 |

*The relative expression level of ADAM19 gene to the expression level of SDHA gene

From Table 2, it was found that the cell population of Example 1 had a higher relative expression level of ADAM19 gene compared with the cell population of Comparative Example 1. It was found that the relative expression level of ADAM19 gene to the expression level of SDHA gene is 3.0 or more (specifically, 4.9) in the cell population of Example 1 while a relative expression level of ADAM19 gene to the expression level of SDHA gene of was less than 3.0 (specifically, 2.3) in the cell population of Comparative Example 1.

### <Evaluation of Angiogenic Capacity>

For the cell populations of the 6th passage cultured in Comparative Example 1 and Example 1, the expressions of ANGPT1, VEGFC, and HBEGF genes, which encode angiogenesis-related cytokines, were analyzed in the same procedures as in the above microarray analysis.

The expression level of each gene was determined as an expression level relative to the expression level of the SDHA gene. The results are shown in the table below.

TABLE 3
Analysis results of angiogenic capacity

| | | ANGPT1 | VEGFC | HBEGF |
|---|---|---|---|---|
| Cell population | Fluorescence | 429.3 | 2205.2 | 2600.0 |
| of Example 1 | intensity | | | |
| | Relative | 0.4 | 2.2 | 2.6 |
| | expression level* | | | |
| Cell population of | Fluorescence | 42.6 | 889.9 | 1170.3 |
| Comparative | intensity | | | |
| Example 1 | Relative | 0.0 | 0.8 | 1.1 |
| | expression level* | | | |

*The relative expression level of each gene to the expression level of SDHA gene

From Table 3, it was found that the cell population of Example 1 had a higher relative expression levels of the respective ANGPT1, VEGFC, and HBEGF genes, compared with the cell population of Comparative Example 1. Consequently, it was found that the cell population of Example 1 had a higher angiogenic capacity compared with the cell population of Comparative Example 1.

From Tables 1 to 3, therefore, the cell population having the following cell characteristics (a) and (b) was found to have high angiogenic capacity:

(a) the proportion of CDH6-positive mesenchymal stem cells is 30% or more in the cell population; and
(b) the relative expression level of ADAM19 gene to the expression level of SDHA gene is 3.0 or more in the cell population.

Furthermore, it was suggested that the conditions of the above (a) and (b) were effective as indices to obtain a cell population having high angiogenic capacity.

### Example 2

“Step 1: Collection of amnion”, “(Step 2: Enzyme treatment of amnion and recovery of amniotic MSCs”, “Step 3: Cryopreservation of amniotic MSCs”, and “Step 4: Culture of amniotic MSCs” were carried out in a manner similar to Example 1, for a pregnant woman (donor) who was an elective cesarean section case after obtaining informed consent (a donor different from Comparative Example 1 and Example 1. A portion of the cell population of each passage was collected during culturing the amniotic MSCs, and then each of the collected cell populations were evaluated for the conditions (a) and (b) below. The evaluation of the conditions was carried out using the same procedures as those of the above sections “Analysis of CDH6 expression” and “Analysis of ADMA19 gene expression”. The conditions are as follows:

(a) the proportion of CDH6-positive mesenchymal stem cells is 30% or more in the cell population; and
(b) the relative expression level of ADAM19 gene to the expression level of SDHA gene is 3.0 or more in the cell population.

The collected cell populations included those satisfying the above conditions (a) and (b) and those not satisfying them. Thus, the two different cell populations were evaluated for angiogenic capacity using the same procedure as “Evaluation of angiogenic capacity” described in paragraph 0119.

As a result, it was found that the cell population satisfying the conditions (a) and (b) had higher relative expression levels of ANGPT1, VEGFC, and HBEGF genes, compared with the cell population not satisfying the conditions (a) and (b). Since the cell population satisfying the conditions (a) and (b) has higher angiogenic capacity compared with the cell population not satisfying the conditions (a) and (b), a cell population having high angiogenic capacity can be selectively obtained by using the conditions (a) and (b) as indices.

### Example 3: Production of Pharmaceutical Composition

A portion of the cell population obtained in the above Example 1 is used for the preparation of a pharmaceutical composition. A pharmaceutical composition (cell preparation) consisting of 4.0×10⁸ amniotic MSCs, 800 mg of HES, 0.7 mL of DMSO, and 20 mL of RPMI1640 medium containing 800 mg of human serum albumin is prepared. The pharmaceutical composition is enclosed in a bag for freezing and preserved in a frozen state. The pharmaceutical composition can be thawed upon use and applied to a patient.

1-8. (canceled)

9. A method for producing a cell population comprising mesenchymal stem cells, the method comprising obtaining a cell population having the following cell characteristics (a) and (b):
(a) the proportion of CDH6-positive mesenchymal stem cells is 30% or more in the cell population; and
(b) the relative expression level of ADAM19 gene to the expression level of SDHA gene is 3.0 or more in the cell population.

10. The method of claim 9, wherein the step of obtaining comprises
treating a sample comprising an extracellular matrix layer collected from a fetal appendage with an enzyme solution to release the mesenchymal stem cells contained in the extracellular matrix layer; and
identifying the cell population having the cell characteristics (a) and (b) from the released mesenchymal stem cells.

11. The method of claim 10, wherein the enzyme solution comprises a collagenase, a metalloproteinase, or both.

12. The method of claim 10 further comprising culturing or cryopreserving the cell population.

13. The method of claim 10, wherein the step of obtaining further comprises selectively separating the identified cell population.

14. The method of claim 9, wherein the cell population has a high angiogenic capacity.

15. The method of claim 9, wherein the cell population is culturable up to 40 days or later after start of an in vitro culture.

16. A cell population comprising mesenchymal stem cells, the cell population having the following cell characteristics (a) and (b):
(a) the proportion of CDH6-positive mesenchymal stem cells is 30% or more in the cell population; and
(b) the relative expression level of ADAM19 gene to the expression level of SDHA gene is 3.0 or more in the cell population.

17. The cell population of claim 16, wherein the cell population is a cultured or cryopreserved cell population.

18. The cell population of claim 16, wherein the mesenchymal stem cells are derived from a fetal appendage.

19. The cell population of claim 16, wherein the cell population has a high angiogenic capacity.

20. The cell population of claim 16, wherein the cell population is culturable up to 40 days or later after start of an in vitro culture.

21. A pharmaceutical composition comprising the cell population of claim 16 and a pharmaceutically acceptable vehicle.

22. The pharmaceutical composition of claim 21 comprising a therapeutically effective amount of the cell population to treat a disease selected from the group consisting of ischemic diseases, lower-limb ischemia, renal ischemia, pulmonary ischemia, ischemic heart disease, coronary heart disease, myocardial infarction, angina pectoris, cardiac failure, cardiomyopathy, valvular disease, cerebrovascular ischemia, stroke, cerebral infarction, intracerebral hematoma, and cerebrovascular paralysis.

23. A method for treating a disease in a subject, the method comprising administering a therapeutically effective amount of the cell population of claim 16 to a subject.

24. The method of claim 23, wherein said subject suffers from or has a disease selected from the group consisting of ischemic diseases, lower-limb ischemia, renal ischemia, pulmonary ischemia, ischemic heart disease, coronary heart disease, myocardial infarction, angina pectoris, cardiac failure, cardiomyopathy, valvular disease, cerebrovascular ischemia, stroke, cerebral infarction, intracerebral hematoma, and cerebrovascular paralysis.

25. The method of claim 23, wherein a single dose of the mesenchymal stem cells to a human is 10⁹ cells/kg body weight or less.

26. The method of claim 23, wherein the cell population is administered by injection.

27. The method of claim 23, wherein the method is for transplanting a cell aggregate or sheet-like structure.
